(19) United States
(12) Patent Application Publication
(10) Pub. No.: US 2006/0200178 A1
(43) Pub. Date: Sep. 7, 2006

(54) Anastomosis device catheter and sheath constructions

(76) Inventors: Kory P. Hamel, Bloomington, MN (US); Vincent G. Copa, Minnetonka, MN (US); Robert L. Rykhus, Edina, MN (US); Suranjan Roychowdhury, Plymouth, MN (US); Sidney F. Hauschild, St. Paul, MN (US); Joseph H. Hofmeister, Minneapolis, MN (US); Eric Vang, Blaine, MN (US)

Correspondence Address: AMS RESEARCH CORPORATION, 10700 BREN ROAD WEST, MINNETONKA, MN, 55343, US

(21) Appl. No.: 11/356,755

(22) Filed: Feb. 17, 2006

Related U.S. Application Data
(60) Provisional application No. 60/654,438, filed on Feb. 18, 2005.

(51) Int. Cl. A61B 17/08

(52) U.S. Cl. 606/153

(57) ABSTRACT

The anastomosis devices of the invention advantageously provide reinforcement structures or components that increase the strength and/or flexibility of the device. These features can be especially beneficial to protect the integrity of the installation of the device within a patient to facilitate the healing process. These features may provide resistance to tearing or ripping of the catheter body or sheath by the tissue approximating structures and/or may provide strength in other areas of the catheter body. The anastomosis devices of the invention may further include method and devices for placing markers on the anastomosis device to facilitate accurate placement of the device within the patient.

### CLAIM FOR PRIORITY

The present application claims priority to U.S. provisional patent application No. 60/654,438, filed Feb. 18, 2005, and entitled “Anastomosis Device Catheter and Sheath Constructions.” The identified provisional patent application is here incorporated by reference.

### TECHNICAL FIELD

The invention relates to devices used for performing anastomosis and other related surgical procedures, including urethral procedures that involve reconnecting urethra and bladder tissues after a radical prostatectomy, vesico-urethral anastomosis, and end-to-end urethral anastomosis.

### BACKGROUND OF THE INVENTION

Anastomosis procedures are required for connecting or re-connecting certain body tissues, e.g., as part of a surgical procedure. The tissues may be part of a body lumen such as a blood vessel, intestinal or other digestive system tissue, or tissues relating to the urinary system. As one example, in a radical prostatectomy, a surgeon removes all or most of a patient's prostate. Because the urethra travels through the prostate immediately before reaching the bladder, the upper part of the urethra is also removed with the surgery. The procedure leaves a severed urethral stump and a severed bladder neck. To restore proper urinary functions, the bladder and the urethra must be reconnected.

Conventionally, a surgeon may execute delicate suturing operations with tiny, fine needles to reconnect these or other anatomical bodies. However, installation of sutures with a needle to connect severed tissues can be a difficult and technique-sensitive task. Many factors can make the task difficult, including a very small amount of tissue to work with (e.g., at the urethral stump and at the bladder neck), and proximal sensitive tissues such as ureters at a bladder and a proximal nerve bundle and sphincter at a urethral stump. These factors result in complicated and delicate suturing procedures that, if not performed properly, could result in complications such as leakage, difficulty in healing or failure to heal, or specific conditions such as incontinence or impotence.

To reduce the risks involved in conventional suturing procedures, anastomosis devices have been developed that include a drainage feature and tissue approximating structure that allow for reconnection of tissues without using traditional sutures. Examples of such anastomosis devices are described, for example, in Applicants' co-pending U.S. patent applications having Ser. No. 10/646,383, filed Aug. 21, 2003, entitled “Anastomosis Device and Related Methods”; Ser. No. 10/919,545, filed Aug. 16, 2004, entitled “Anastomosis Device and Related Methods”; and Ser. No. 10/919,775 filed Aug. 16, 2004, entitled “Anastomosis Device and Related Methods”, all of which are incorporated herein by reference in their entireties. These anastomosis devices advantageously use tissue approximating structures to reconnect severed tissues during anastomosis procedures, which can both reduce the risks during the surgical procedure and also provide a significant reduction in the amount of time required to perform certain anastomosis procedures. Because the anastomosis device will typically be surgically positioned within the patient for a significant period of time (e.g., while the healing process takes place), there is a need for the device to be sufficiently strong and flexible to accommodate the various stresses to which the device may be subjected while positioned within the patient.

### SUMMARY OF THE INVENTION

Anastomosis devices and related surgical tools and external connecting devices of the invention preferably include an elongated body, tissue approximating structures that extend from the elongated body, such as one or multiple sets of tines, mechanisms for actuating the tissue approximating structures, a drainage lumen that may extend as a channel through the length of the elongated body and that communicates at its distal end with a drainage aperture, and a balloon at or adjacent to the distal end of the device. The actuating mechanisms for the tissue approximating structure are located generally near a proximal end of the device and may include any of a wide variety of actuation configurations that can provide for extension and retraction of the tissue approximating structure, as desired. The reinforcement configurations described herein may apply to any of these types of anastomosis devices that may remain within a patient for a significant time period due to the use of the tissue approximating structures. However, many of the embodiments of the invention may also apply to anastomosis devices that are differently configured, such as a device that does not include a balloon, for example.

One exemplary configuration for the tissue approximating structure includes two sets of tines spaced from each other along the length of the catheter body that can be extended and retracted from the catheter body. In this example embodiment, each of the sets of tines is controlled by an actuation mechanism that is attached to the anastomosis device and is positioned outside the patient's body. Each of the sets of tines may be simultaneously or sequentially moveable, as desired.

The anastomosis devices of the invention advantageously provide reinforcement structures or components that increase the strength and/or flexibility of the device. These features can be especially beneficial to protect the integrity of the installation of the device within a patient to facilitate the healing process. These features may provide resistance to tearing or ripping of the catheter body or sheath by the tissue approximating structures and/or may provide strength in other areas of the catheter body.

The anastomosis devices of the invention may further include method and devices for placing markers on the anastomosis device to facilitate accurate placement of the device within the patient.

### BRIEF DESCRIPTION OF THE DRAWINGS

The invention will be further explained with reference to the appended Figures, wherein like structure is referred to by like numerals throughout the several views, and wherein:

FIG. 1a is a perspective view of an anastomosis device including a cable or wire within the catheter shaft;

FIG. 1b is a cross-sectional end view of the device of FIG. 1a;

FIG. 1c is a cross-sectional front view of a portion of an anastomosis device having a reinforcing structure bonded within a lumen of the device;

FIG. 2 is a perspective view of a portion of an anastomosis device including a strain relief feature;

FIG. 3 is a cut-away perspective view of the device of FIG. 2;

FIG. 4a is a perspective view of a tip portion of an anastomosis device including an integrated mesh in a connective sheath;

FIG. 4b is a top view of another configuration of mesh in a portion of an anastomosis device;

FIG. 5 is a perspective view of a tip portion of an anastomosis device similar to that of FIG. 4a, but including a larger mesh area;

FIG. 6a is a perspective view of a tip portion of an anastomosis device including reinforcement extension areas;

FIG. 6b is a top view of the device of FIG. 6a;

FIG. 7a is a perspective view of a tip portion of an anastomosis device having a force-transmitting inner construction;

FIG. 7b is a schematic top view of the inner area of a portion of the tip of FIG. 7a.

### DETAILED DESCRIPTION OF THE PREFERRED EMBODIMENTS

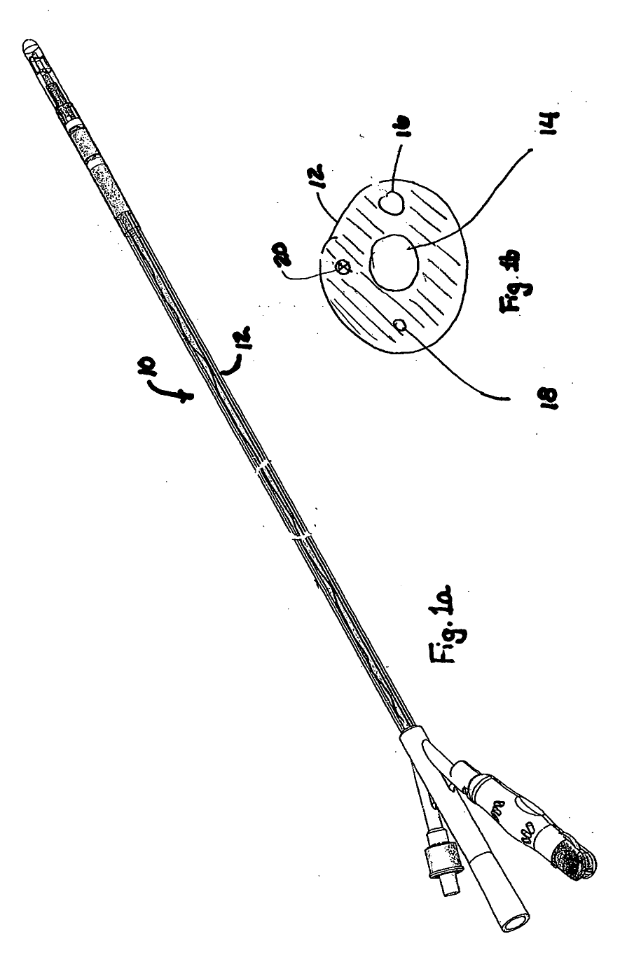

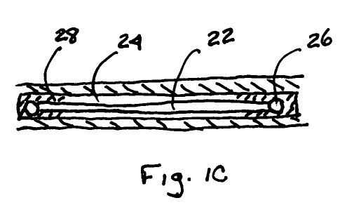

Referring now to the Figures, wherein the components are labeled with like numerals throughout the several Figures, and initially to FIGS. 1a and 1b, one exemplary embodiment of an anastomosis device 10 is illustrated, which includes an elongated catheter shaft 12 having a central drainage lumen 14 that extends generally along the length of catheter shaft 12. Catheter shaft 12 further includes an actuation wire lumen 16 through which an actuation wire for the tissue approximating structure can extend, and an inflation lumen 18 used for inflation and deflation of a balloon near the tip of the device. Catheter shaft 12 further includes at least one cable or wire 20 that acts as a reinforcement structure and extends through the wall of catheter shaft 12 along at least part of its length. This cable or wire 20 is made of a material that provides added strength to the device 10 to prevent or minimize stretching or deformation of catheter shaft 12 during manipulation of device 10 and/or to protect the integrity of the device if it is subjected to unanticipated loads, such as impact loads. Thus, cable or wire 20 is preferably chosen or designed to be as thin and lightweight as possible so as to not add unnecessary weight or bulk to device 10, while still providing the desired amount of protection for the device. In addition, cable or wire 20 should be of a size and shape that maintains a relatively flexible catheter shaft 12 for patient comfort. More than one cable or wire 20 may be used in a particular catheter shaft 12, where multiple wires 20 within a single device may be spaced from each other within the wall of catheter shaft 12. Cable or wire 20 may be coextruded within the wall of catheter shaft 12, or may instead extend through a central opening of catheter shaft 12. In a further alternative, cables or wires 20 may be located on the outside of catheter shaft 12 and may be secured to shaft 12 by adhesive mechanical attachment, wrapping of wire 20 about shaft 12, and the like. Cables or wires 20 may be solid lengths of material such as metal, plastic, fabrics, woven materials, and the like, or may instead be hollow tubular structures. Each cable or wire 20 may comprise more than one piece of the same or different materials that are woven or otherwise attached to each other along their lengths.

FIG. 1c is a cross-sectional view of a section of wire or cable 22 that may be have a structure as described above relative to FIGS. 1a and 1b. Wire or cable 22 extends through a lumen 24 along at least a portion of a catheter shaft, and may optionally include a ball or enlarged portion 26 at one or both of its ends. This embodiment further includes an adhesive 28 within at least a portion of lumen 24 for securing wire 22 so that it cannot move relative to lumen 24. If wire 22 includes an enlarged portion 26, this enlarged portion 26 can provide an anchor point to help prevent movement of wire 22 along the length of the catheter shaft. Adhesive 28 may optionally be provided in a color or material that is visible or otherwise detectable to determine the amount of adhesive 28 placed within lumen 24. In this way, the manufacturer can detect when an adequate amount of adhesive 28 is added to lumen 24, which can also prevent overflow of adhesive from lumen 24 that may occur with the addition of too much adhesive.

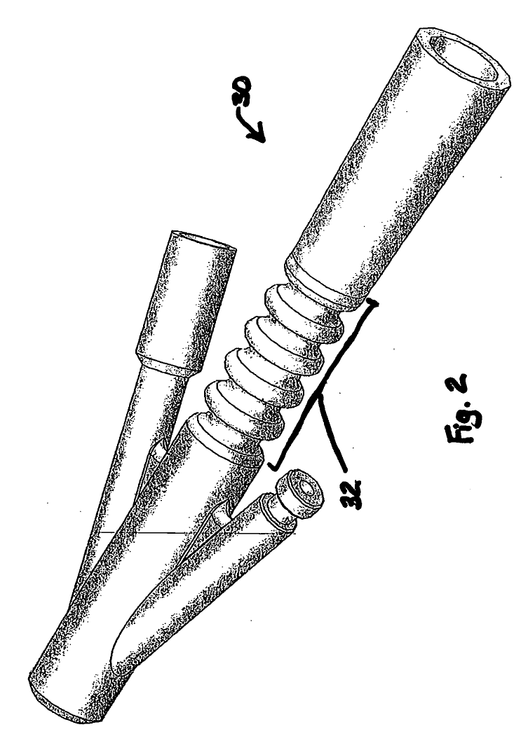

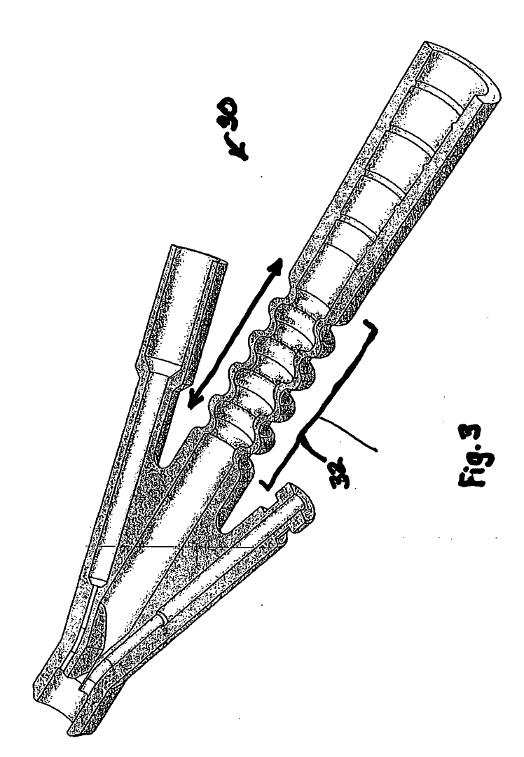

FIGS. 2 and 3 illustrate a catheter funnel 30 of an anastomosis device that would typically be located at the proximal end of the anastomosis device. This funnel 30 includes an integrated strain relief portion 32 that allows for compression and extension of funnel portion 30 when the device is subjected to external loads. Relief portion 32 is illustrated as an accordion-like section of the device between two sections that are generally not expandable or compressible. Relief portion 32 may be as long or short as desired, where a longer relief portion 32 will provide additional flexibility to the device. Relief portion 32 should have sufficient radial strength to keep it from collapsing on itself or kinking during or after placement within a patient. In addition, relief portion 32 should be long enough that it allows for a normal amount of compression or extension of the device when subjected to loads. Relief portion 32 may be a portion of funnel 30 that is an integral to funnel 30 (e.g., it is integrally molded or formed with the other portions of funnel 30). Alternatively, relief portion 32 may be a separate section that is attached at one or both ends to remainder of funnel 30. Further, relief portion 32 may be provided in a different configuration than that shown in order to provide a different flexibility for this portion as compared to the portions on either side of it. For example, relief portion 32 may be configured as an elongated tube that is made of a material that is more flexible or elastic than the areas of funnel 30 to which it is attached, thereby providing the desired strain relief properties to funnel 30.

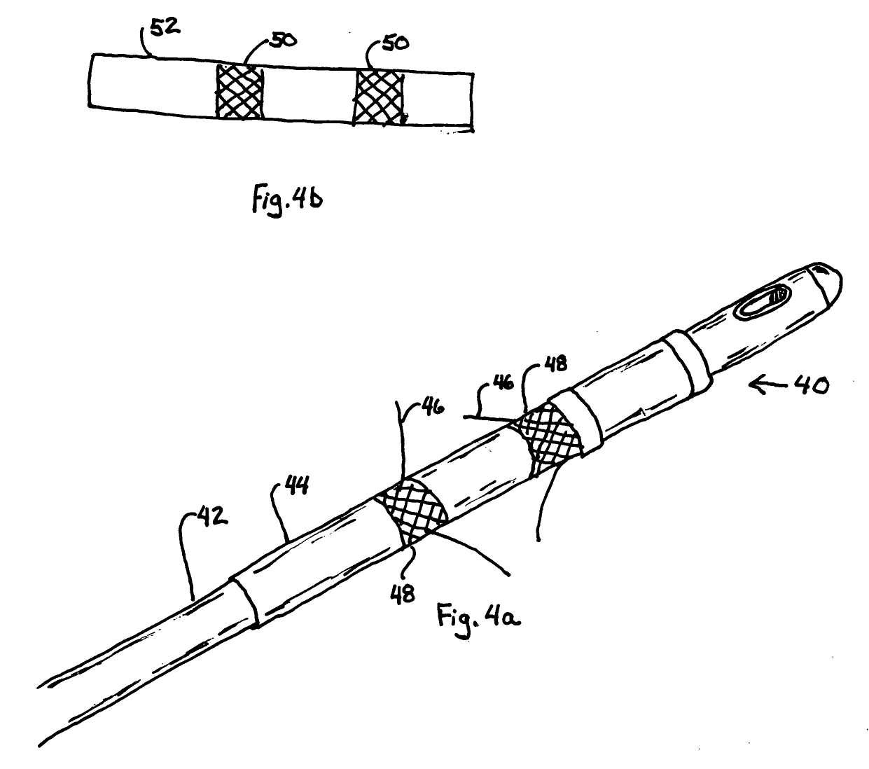

Referring now to FIG. 4a, a tip portion 40 of an anastomosis device is illustrated, which includes a catheter shaft 42 and a plurality of extending tissue approximating structures or tines 46. Tip portion 40 further includes a connective sheath 44 having two rings of mesh 48 in the general area of the extending tines 46. Rings of mesh 48 are spaced from each other along the length of sheath 44, and are preferably positioned to include most or all of the areas of sheath 44 through which tines 46 may extend. That is, if the device includes two sets of tines 46, one ring of mesh 48 is preferably provided for each set of tines 46, although it is possible that less rings of mesh 48 are provided than the number of sets of tines 46. Mesh 48 adds support to the structure in the areas where the tines extend through sheath 44 and thus may be provided in any area of the anastomosis device through which tines extend (e.g., through the catheter shaft if this is the area of the device that includes extending tines). In this way, the areas of mesh 48 can prevent or minimize tearing or other damage to sheath 44 or catheter body that may occur if it is subjected to certain forces that are placed on the anastomosis device. In addition, mesh 48 can provide sufficient additional structure to the device to prevent sheath 44 from stretching. Mesh 48 may be integrated into connective sheath 44, as shown in FIG. 4a. Alternatively, mesh portions 50 may be overmolded or bonded to a connective sheath, such as to the portion of a connective sheath 52 illustrated in FIG. 4b. Sheaths 44, 52 may be made of a flexible material, such as silicone, and mesh portions 48, 50 are preferably made of a relatively lightweight and bendable material, such as metal, plastic, natural or synthetic fibers, or the like. Mesh portions 48, 50 may also have a wide variety of configurations, such as a woven or knitted construction, that allow for relatively easy penetration of any tissue approximating structures.

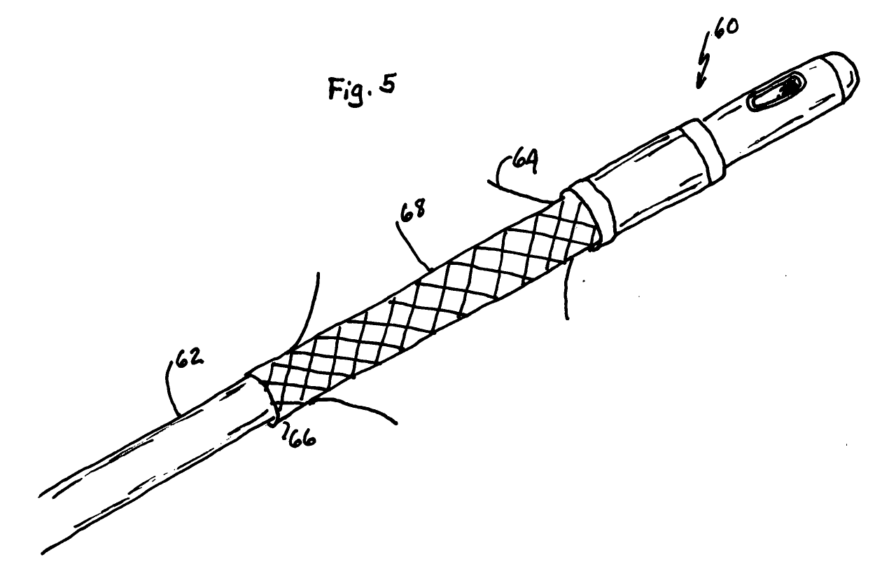

FIG. 5 illustrates another tip portion 60 of an anastomosis device, which includes a catheter shaft 62 and a plurality of extending tissue approximating structures or tines 64. Tip portion 60 further includes a connective sheath 66 having a single elongated area of mesh 68 that extends along the length of sheath 66 for a distance that is long enough to encompass all of the sets of tines 64 that extend from sheath 66. In this embodiment, mesh 68 extends along the entire length of sheath 66, which thereby includes both sets of tines 64. As with the mesh reinforcements provided in FIGS. 4a and 4b, mesh 68 preferably provides sufficient additional structure to the device to prevent sheath 66 from stretching, and also preferably is strong enough to prevent or minimize tearing or other damage to sheath 66 or catheter body that may occur if it is subjected to certain forces that are placed on the anastomosis device. Mesh 68 may either be incorporated into connective sheath 66 or be bonded or overmolded to connective sheath 66. Further, mesh 68 may be provided in any area of the anastomosis device through which tines extend (e.g., through the catheter shaft or some other component of the device if this is the area of the device that includes extendible tines).

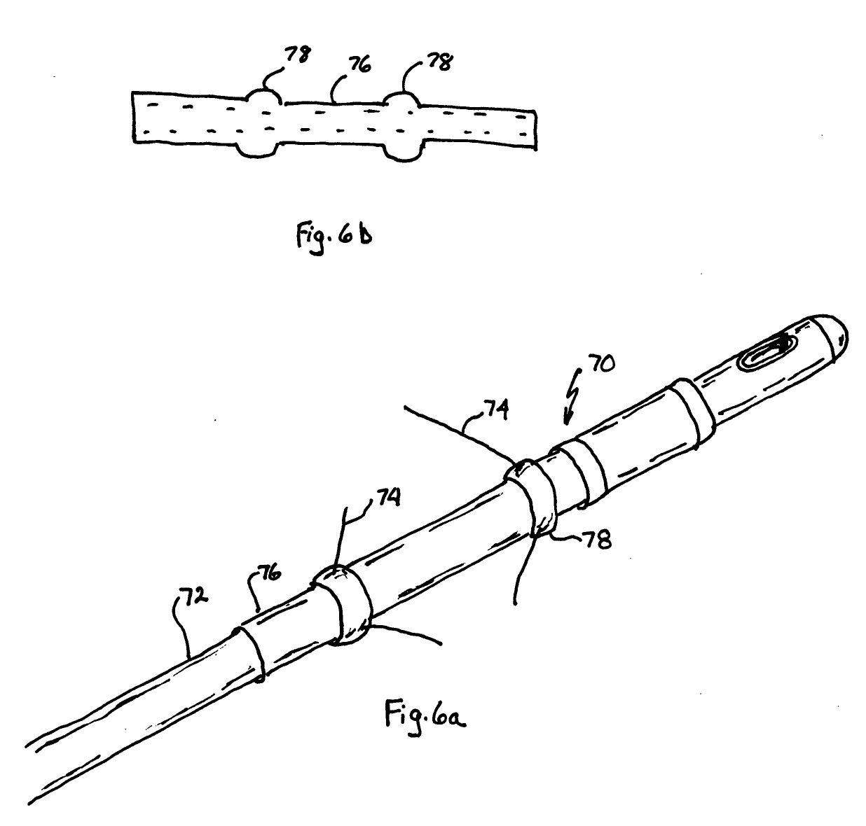

Another tip portion 70 of an anastomosis device is illustrated in FIGS. 6a and 6b, which includes a catheter shaft 72 and a plurality of extending tissue approximating structures or tines 74. Tip portion 70 further includes a connective sheath 76 having reinforced portions 78 in the area through which tines 74 extend from its outer surface. As shown, reinforced portions 78 may be extending rings of material that are thicker than surrounding sheath 76. The height, width, and thickness of portions 78 may be similar or different from the illustrated portions 78, but should preferably provide a desired additional strength to the sheath that helps to prevent or minimize tearing or other damage to sheath 76 in the areas where tines 74 extend through sheath 76. Portions 78 may either be incorporated into the connective sheath 76 or be bonded or overmolded to connective sheath 76. Again, the reinforced portions may be provided in any area of the anastomosis device through which tines extend, such as through the catheter shaft if this is the area of the device that includes extendible tines.

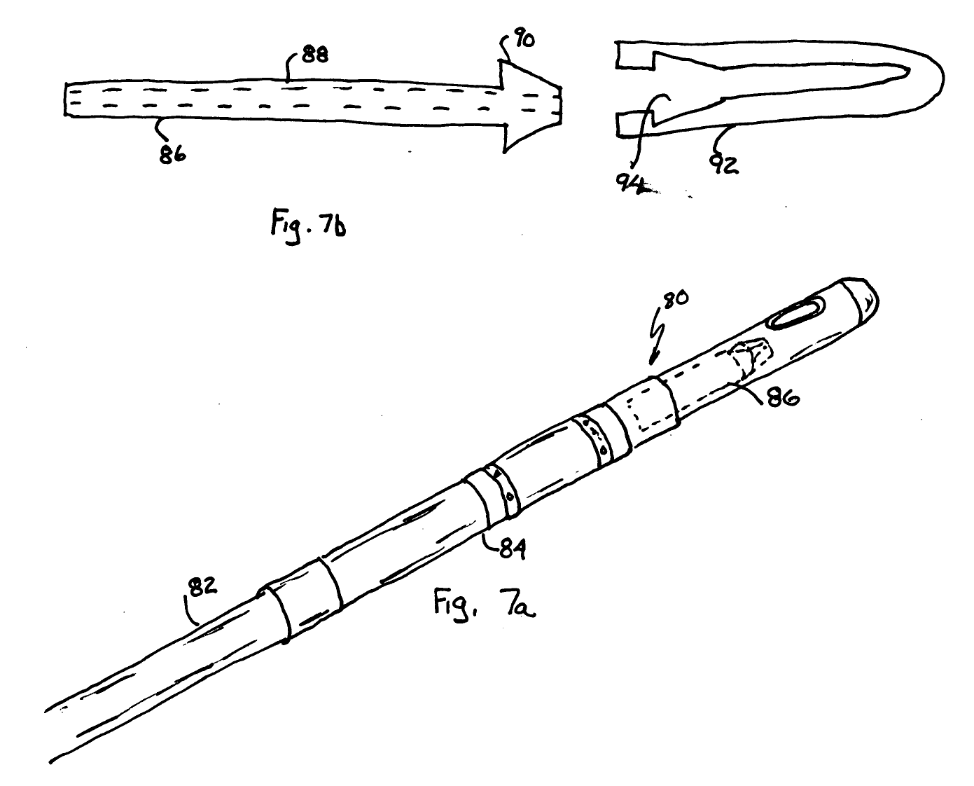

FIG. 7a illustrates a tip portion or assembly 80 of another embodiment of an anastomosis device, which includes a catheter shaft 82 and a connective sheath 84. Tip portion 80 comprises a flexible core 86 (also illustrated in FIG. 7b) configured as an elongated tubular portion 88 with a flanged tip 90 at one end. Flanged tip 90 is designed to mate or interlock with an opening 94 of a tip 92. In this way, force that is exerted on the bottom of a balloon when it is inside the patient will be transmitted along the length of tip assembly 80 and back to catheter shaft 82. This transmission of force can therefore minimize the forces on the portions of assembly 80 that can include tissue approximating structures or tines, for example. Flexible core 86 may be configured with differently sized and shaped tubular portions and flanged tips to provide a similar force transmission. Of course, the tip would need to be designed to mate with the portions of the flexible core that would be inserted within it.

The anastomosis devices of the invention may further include methods and devices for placing markers on the anastomosis device to facilitate accurate placement of the device within the patient. For one example, tissue approximating structure markers can be used to indicate the location from where the tissue approximating structures expand or extend. These markers could be differentiated visually, such as with color, radiographically, echogenically, or the like. For another example, luminal markers can be used to indicate the locations of the various lumens within a device. These markers could also be differentiated visually, such as with color, radiographically, echogenically, or the like. For another example, directional markers can be used to indicate if and when the tissue approximating structures have been deployed or retracted. These markers could be visual, tactile, audible, or detectable in any other manner. These directional markers can also correspond to the color of other areas of the device, such as the areas where the tine sets are located, so that the user can determine which of multiple sets of tines are deployed or retracted at a given time.

A particular anastomosis device may include one or a combination of the structures and devices described above to reinforce or otherwise enhance the flexibility, strength or other properties of a device, as desired. The choices may be made to protect the device from the most likely forces or strains that may occur, thereby preventing or minimizing damage to the anastomosis device and/or injury or discomfort to the patient.

In a related embodiment, a surgical tool is disclosed that is adapted to include the strain relief feature described above for connecting external devices to external tissues of a human or animal body. The strain relief features described herein are also applicable to, but not limited to, other external communicating medical devices or tools that may include Foley catheters, dialysis ports, venous access devices, and the like.

The invention has now been described with reference to several embodiments thereof. The foregoing detailed description and examples have been given for clarity of understanding only. No unnecessary limitations are to be understood therefrom. It will be apparent to those skilled in the art that many changes can be made in the embodiments described without departing from the scope of the invention.

What is claimed:

1. A surgical tool comprising:
an elongated body having a proximal end and a distal end;
at least one set of tissue approximating structures that are extendible through and retractable from the elongated body;
an actuating mechanism in operable communication with the at least one set of tissue approximating structures to extend and retract the at least one set of tissue approximating structures;
a drainage lumen extending from a drainage aperture proximate the distal end of the elongated body to a drainage port; and
wherein the elongated body includes a reinforcing cable or wire extending along at least a portion of the elongated body.

2. The surgical tool of claim 1, wherein the elongated body includes a plurality reinforcing cables or wires extending along at least a portion of the elongated body.

3. The surgical tool of claim 1, wherein said reinforcing cable or wire is coextruded with the elongated body.

4. The surgical tool of claim 1, wherein said reinforcing cable or wire extends through a central opening of the elongated body or a lumen of the elongated body.

5. The surgical tool of claim 1, wherein said reinforcing cable or wire is secured to an exterior surface of the elongated body.

6. The surgical tool of claim 4, wherein said reinforcing cable or wire includes an anchor portion at its proximal and/or distal end.

7. The surgical tool of claim 6, further comprising adhesive within the central opening or lumen to secure to the reinforcing cable or wire.

8. A surgical tool comprising:
an elongated body having a proximal end and a distal end;
at least one set of tissue approximating structures that are extendible through and retractable from the elongated body;
an actuating mechanism in operable communication with the at least one set of tissue approximating structures to extend and retract the at least one set of tissue approximating structures;
a drainage lumen extending from a drainage aperture proximate the distal end of the elongated body to a drainage port; and
wherein the elongated body comprises at least one strain relief portion.

9. The surgical tool of claim 8, wherein the at least one strain relief portion comprises an accordion strain relief portion.

10. The surgical tool of claim 8, wherein the at least one strain relief portion is intermediate the elongated body and wherein the at least one strain relief portion is of a more flexible or elastic material than the material of the elongated body by which the strain relief portion is bounded.

11. The surgical tool of claim 8, wherein said elongated body includes a protective sheath, wherein the strain relief portion is a portion of said protective sheath, and wherein the strain relief portion comprises a mesh portion, wherein said mesh portion is located in the general area of the at least one set of tissue approximating structures, and wherein the at least one set of tissue approximating structures extends through said mesh portion upon being extended.

12. The surgical tool of claim 11, wherein said mesh is unitary with said protective sheath.

13. The surgical tool of claim 11, wherein said mesh is non-unitary with said protective sheath.

14. The surgical tool of claim 8, wherein said elongated body includes a protective sheath, wherein the strain relief portion is a portion of said protective sheath, and wherein the strain relief portion comprises an extended portion having a thickness greater than the thickness of the protective sheath, wherein said extended portion is located in the general area of the at least one set of tissue approximating structures, and wherein the at least one set of tissue approximating structures extends through said extended portion of said protective sheath upon being extended.

15. The surgical tool of claim 14, wherein the extended portion is unitary with said protective sheath.

16. The surgical tool of claim 14, wherein the extended portion is non-unitary with said protective sheath.

17. An anastomosis device comprising:
an elongated body having a proximal end and a distal end;
at least one set of tissue approximating structures that are extendible through and retractable from the elongated body;
an actuating mechanism in operable communication with the at least one set of tissue approximating structures to extend and retract the at least one set of tissue approximating structures;
a drainage lumen extending from a drainage aperture proximate the distal end of the elongated body to a drainage port; and
wherein the elongated body comprises at least one placement marker for detection during the placement of the anastomosis device within a patient.

18. The anastomosis device of claim 17, wherein the at least one placement marker for detection comprises a tissue approximating structure marker, a luminal marker, or a directional marker.

19. The anastomosis device of claim 18, wherein the tissue approximating structure marker or luminal marker are differentiated visually with color, radiographically, or echogenically.

20. The anastomosis device of claim 18, wherein the directional marker is selected from a group consisting of: a visual marker, a tactile marker or an audible marker.
